(19) United States
(12) Patent Application Publication
(10) Pub. No.: US 2005/0271754 A1
(43) Pub. Date: Dec. 8, 2005

(54) Composition for prevention or treatment of an alcohol hangover

(76) Inventor: Patrick W. Cochrane, San Diego, CA (US)

Correspondence Address: RICHARD D. CLARKE;LAW OFFICE OF RICHARD D. CLARKE, 3755 AVOCADO BLVD., #1000, LA MESA, CA, 91941-7301, US

(21) Appl. No.: 11/146,747

(22) Filed: Jun. 6, 2005

Related U.S. Application Data
(60) Provisional application No. 60/577,778, filed on Jun. 7, 2004.

(51) Int. Cl. A61K 38/05; A61K 35/78; A61K 31/4415; A61K 31/714; A61K 31/198; A61K 31/525; A61K 31/19

(52) U.S. Cl. 424/750; 514/18; 514/52; 514/251; 514/275; 514/350; 514/562; 514/574

(57) ABSTRACT

The present invention represents a formulation and method for preventing or reducing the effects of veisalgia or alcohol “hangover.” More particularly, the present invention relates to a treatment which provides nutritional requirements in the form of L-glutamine, L-cysteine, fumaric acid, succinic acid, young barley grass juice powder, vitamin B-12, vitamin B-1, calcium ascorbate, and dextrose. The composition may be ingested before, before and during, or after consumption of ethyl alcohol. The composition of the present invention is made available in several forms including, but not limited to, capsule, tablet or liquid form suitable for administration for amelioration of alcohol induced hangover symptoms. This treatment enables rapid relief of symptoms in affected individuals by slowing or reducing the conversion of ethanol into acetaldehyde, a toxic substance resulting from alcohol metabolism. Additionally, the present invention also incorporates nutritional elements which are directed toward assisting in the conversion of acetaldehyde into acetic acid and the oxidative processes in the mitochondria responsible for decomposition of acetaldehyde.

This application claims the benefit of U.S. provisional patent application Ser. No. 60/577,778, filed on Jun. 7, 2004.

### BACKGROUND OF THE INVENTION

1. Field of the Invention

The present invention relates to nutritional remedial compound for use in the prevention of, or treatment of symptoms of alcohol induced toxicity known as veisalgia, or more commonly referred to as “hangover,” and the method of making and using same. More particularly, the present invention relates to a composition formulated from L-glutamine, L-cysteine, fumaric acid, succinic acid, young barley grass juice powder, vitamin B-12, vitamin B-1, calcium ascorbate, and dextrose. When combined and properly administered to affected individuals suffering from symptoms of hangover, the present composition acts to rapidly relieve the symptoms associated with alcohol toxicity known as hangover.

2. Description of the Related Art

Consumption of alcoholic beverages causes the syndrome known as hangover. Hangover effects are unpleasant and vary in intensity and symptoms by individuals. The most common hangover effects experienced are headache, nausea, dizziness, fatigue, thirst, tension, anxiety, paleness, tremor and perspiration. Prevention and amelioration of some or all of these symptoms is desirable not only from an individual standpoint, but also from the concern of employers and society in general, since productive time is lost by individuals suffering therefrom.

When ethanol is consumed, it is absorbed by the small intestine and carried throughout the body via the bloodstream. As the alcohol passes through the liver, it is metabolized into acetaldehyde, Eventually, the enzyme acetaldehyde dehydrogenase converts the acetaldehyde into acetic acid (vinegar), a harmless chemical, but not before the lingering acetaldehyde in the organs and tissues causes increased heart rate, nausea, headache, queasiness, flushing, and other classic hangover-related symptoms.

Many individuals are very slow or unable to metabolize acetaldehyde into acetic acid, and therefore have a lower tolerance to alcohol and are more likely to suffer more severe hangover symptoms due to the toxic effects of acetaldehyde.

A condition known as “Asian flush” occurs in 45% of the Chinese and Japanese populations whereby a genetic deficiency of the enzyme aldehyde dehydrogenase causes them to have problems converting the acetaldehyde into harmless acetic acid and therefore toxic levels of acetaldehyde build up in their systems resulting in severe and immediate hangover symptoms.

The benefits of compounds designed for ameliorating the symptoms of hangover are well known. Examples of different types and kinds of compositions for treatment of symptoms of hangover are disclosed in U.S. Pat. Nos. 6,713,091 and 4,496,548.

Compositions for lowering the concentration of alcohol in blood are known in the prior art. Such a treatment is described in U.S. Pat. No. 6,713,091. This inventive formulation includes extracts of the pepino leaves, stalk and fruit and licorice. The formulation specifically lowers levels of alcohol in the blood with ingestion of the extracts prior to ingestion of alcohol.

While in vivo tests indicated lower alcohol levels in blood activity, it is unclear from studies as to the effectiveness of the novel compound in treatment of symptoms of alcohol toxicity when taken after alcohol consumption. It is also unclear as to the length of time from the beginning of treatment to the relief of symptoms from the information provided.

In addition, the availability of the pepino plants used for this inventive formulation is not addressed. If the pepino plants are relatively rare, this formulation would be difficult to produce and might be cost prohibitive to duplicate.

There is no information addressing the palatability of the compound or any adverse affects to the digestive system.

Therefore, it would be highly desirable to have a new and improved composition for use in treatment of hangover symptoms which would allow rapid relief from the symptoms, which could be taken prior to, during or after alcohol consumption and be effective in reducing symptoms associated with alcohol toxicity related hangover. Furthermore, it would be highly desirable that such a compound be made from readily available ingredients and be economically feasible to produce.

The treatment described in U.S. Pat. No. 4,496,548 addresses the problem of providing for a formulation which would be useful for treatment of hangover before during or after alcohol consumption but is especially suitable for prevention of hangover by administration before alcohol consumption. Since consumption of alcoholic beverages is largely a social behavior, there is often spontaneous consumption with unpredictable levels of alcohol consumed so ingestion prior to consumption of alcoholic beverages may be impractical.

Therefore, it would be highly desirable to have a new and improved formulation and method for using same for a composition for treatment of an alcohol related hangover which would be useful and effective if ingested prior to, during or after alcohol consumption which would provide relief from the symptoms of an alcohol related hangover.

### SUMMARY OF THE INVENTION

Therefore, the principal advantage of the present invention is to provide a new and improved formulation and method of using same, for a composition for reducing the symptoms of alcohol-induced hangover, that would be effective if taken prior to, during or after alcohol consumption.

It is a further advantage of the present invention to provide such a new and improved formulation and method for using same, for a composition for reducing the symptoms of alcohol related hangover that is relatively inexpensive to produce and relieves symptoms within a very short period of time with little or no toxicity for the person being treated.

It is yet a further advantage of the present invention to provide such a new and improved formulation and method for using same, for a composition for reducing the symptoms of alcohol related hangover, which would be formulated from readily available ingredients. The formulation of the treatment provides a simple, yet effective means by which to treat affected individuals.

Briefly, the above and further advantages of the present invention are realized by providing a new and improved formulation and method for using same, composition for reducing the symptoms of alcohol related hangover. More particularly, the present invention relates to a treatment which enables rapid relief of symptoms with little or no toxicity exhibited for the affected individual.

### DETAILED DESCRIPTION OF THE CERTAIN EMBODIMENTS

The treatment for alcohol induced hangover of the present invention is comprised in a liquid state as a capsule, tablet, powder or liquid form to support detoxification and elimination of alcohol from the body.

The treatment provides nutritional requirements in the form of at least one component based on succinic acid, at least one component based on fumaric acid, at least one sub component of the tripeptide glutathione L-glutamine, at least one component based on L-cysteine, at least one component based on young barley grass juice powder, folic acid, a component based on Vitamin B-12, a component based on Vitamin B-1 thiamine hydrochloride, a component based on calcium ascorbate and one energy source from the group of dextrose, fructose, glucose.

The recommended dosage of the present powder composition is approximately two 500 mg capsules for every two alcoholic beverages consumed. This remedy may also be consumed to relieve the hangover symptoms when taken a number of hours following the consumption of alcohol.

Examples of compositions for preparing the present powder composition of the invention for reducing the symptoms of alcohol related hangover are given to illustrate the composition and are not limited to the examples shown.

### EXAMPLE 1

A capsule of the present powder composition comprises:
250 mg. of dextrose
250 mg. of succinic acid
250 mg. of L-glutamine
100 mg. of fumaric acid
50 mg. of calcium ascorbate
75 mg. of young barley grass juice powder
15 mg. of L-cysteine as L-cysteine HCl
2 mg. of Vitamin B1 as thiamine hydrochloride
1200 mcg. of Vitamin B12 as cyanocobalamin
400 mcg. of folic acid

### EXAMPLE 2

A pressed tablet of the present powder composition comprises:
200 mg. of dextrose
200 mg. of succinic acid
200 mg. of L-Glutamine
75 mg. of fumaric acid
50 mg. of calcium ascorbate
50 mg. of young barley grass juice powder
50 mg. of L-Cysteine as L-cysteine HCl
2 mg. of Vitamin B1 as thiamine hydrochloride
1200 mcg. of Vitamin B12 as cyanocobalamin
400 mcg. of folic acid

### EXAMPLE 3

A 1 oz. serving size of the present liquid composition comprises:
10 to 500 mg. of fumaric acid
10 to 400 mg. of succinic acid
10 to 500 mg. of L-glutamine
10 to 400 mg. of dextrose
10 to 200 mg. of sodium ascorbate
10 to 100 mg. of sodium benzoate
10 to 100 mg. of disodium phosphate
10 to 100 mg. of sodium citrate
10 to 100 mg. of sodium chloride
10 to 100 mg. of potassium phosphate
10 to 100 mg. of monopotassium phosphate
10 to 100 mg. of sucrose acetate isobutyrate
10 to 200 mg. of niacinamide
10 to 200 mg. of pyridoxine hydrochloride
5 to 100 g of purified water

With administration of the powder form of the composition, the capsules or tablets can be taken orally before, during or after consuming alcoholic beverages. Typical doses range from one to two 100 mg tablets to one to two 500 mg capsules per every two alcoholic beverages consumed, or as needed.

With administration of the liquid form of the composition, a 1 oz. serving (a typical “shot” size) per every two alcoholic beverages consumed, is taken, or as needed. The composition is most effective when taken during alcohol consumption, but may also be effective after alcohol consumption when taken prior to sleep, or even the morning after consuming alcoholic beverages. The powder (capsules and tablets) and liquid forms of the composition may also be taken prior to alcohol consumption with some effectiveness. The present composition will not lower the blood alcohol level nor will it prevent intoxication.

When an alcoholic beverage is consumed, the alcohol in the drink (ethanol) is absorbed in the stomach and passes into the portal vein and then goes into the liver (the body's filtration organ) where the liver starts to metabolize the ethanol.

Ethanol (CH₃CH₂OH) is oxidized with the enzyme alcohol dehydrogenase (ADH) in the presence of the co-enzyme nicotinamide adenine dinucleotide (NAD⁺) and converted into Acetaldehyde. The NAD⁺ is converted into nicotinamide adenine dinucleotide in reduced form (NADH) and H⁺.

Acetaldehyde (CH₂CHO), an aldehyde, is a toxic substance that is then oxidized by the co-enzyme nicotinamide adenine dinucleotide (NAD⁺) in the presence of the enzyme aldehyde dehydrogenase 2 (ALDH2) along with water and is converted into the harmless chemical acetic acid. Acetic acid is then converted to acetyl CoA by a thiokinase enzyme.

When the levels of acetaldehyde exceed that which the liver is able to process into acetic acid, the acetaldehyde dumps over into the general blood circulation and leads to hangover symptoms. Acetaldehyde causes a whole range of symptoms by virtue of the fact that it is a potent nerve irritant and neurotoxin.

The ADH and ALDH2 catalyzed reactions lead to the reduction of NAD⁺ to NADH. The metabolic effects of ethanol intoxication may also stem from the actions of ADH and ALDH2 and the resultant cellular imbalance in the NADH/NAD⁺. The NADH produced in the cytosol by ADH must be reduced back to NAD⁺ via either the malate aspartate shuttle or the glycerol-phosphate shuttle.

Thus, the ability of an individual to metabolize ethanol is dependent upon the capacity of hepatocytes to carry out either of these two shuttles which in turn is affected by the rate of the TCA cycle in the mitochondria whose rate of function is being impacted by the NADH produced by the ALDH2 reaction.

In theory to combat hangover the conversion of ethanol into acetaldehyde by the enzyme alcohol dehydrogenase would need to be slowed down to a level where the liver could keep pace converting it into acetic acid, and/or blocked, and/or the process of oxidation of acetaldehyde into acetic acid would need to be accelerated.

The body is only able to metabolize a certain amount of acetaldehyde at a time naturally so it is apparent that unless intervention takes place that the liver becomes overwhelmed with large amounts of acetaldehyde and a toxic situation, veisalgia, or hangover, ensues.

Human females possess slightly lower levels of the aldehyde dehydrogenase enzyme than their male counterparts.

A condition known as “Asian flush” occurs in 45% of the Chinese and Japanese populations, who possess non-functional copies of the enzyme aldehyde dehydrogenase-2, which causes them to have difficulty converting the acetaldehyde into harmless acetic acid and therefore toxic levels of acetaldehyde build up in their systems resulting in severe and immediate hangover symptoms.

The body also has the ability to deploy an aldehyde-scavenging defense against acetaldehyde using the amino acid cysteine and a peptide known as glutathione. These molecules contain sulfur compounds that are chemically active in combating aldehydes.

Oxidized sulfur compounds are inactive against aldehydes so to keep cysteine from being oxidized and able to combat aldehydes, there must be a presence of vitamin C in a 3 to 1 ratio.

The present invention combats hangover symptoms by attacking the problem from three angles.

The present invention slows and or reduces the conversion of ethanol into acetaldehyde. Reducing the amount of acetaldehyde produced is accomplished by the inclusion of succinic acid and fumaric acid to activate the second half-cycle of tricarboxylic acids and accelerate the decomposition of acetaldehyde and energization of the oxidation processes in the mitochondria.

Barley grass juice powder is added to the formula. The barley grass juice powder has been found to contain a unique bioflavonoid called 2″-O-Glycosylisovitexin or 2″-O-GIV which inhibits the formation of acetaldehyde.

Dextrose is added to the formula to compete with ethanol for available cytosol NAD thereby creating a deficit of NAD needed by ethanol to convert into acetaldehyde.

The present invention speeds up the conversion of acetaldehyde into acetic acid. L-cysteine is included as it was found to increase the oxidation cycle of ethanol lowering acetaldehyde levels in the blood, liver and brain.

L-glutamine (C₅H₁₀N₂O₃) has been added to speed up the process of reducing the NADH back to NAD⁺ via the malate-aspartate shuttle to correct the cellular imbalance in the NADH/NAD⁺, believed to be one of the metabolic effects of ethanol intoxication.

The present invention scavenges unmetabolized acetaldehyde. Research has shown that antioxidants containing a form of sulfur called a sulfhydryl group are effective in scavenging acetaldehyde that is not metabolized into acetic acid. Two important antioxidants in this group are cysteine and glutathione. Glutathione and cysteine are aldehyde scavengers and can be used by the body to mop up acetaldehyde. The current formulation includes L-Cysteine, one such antioxidant, and the glutathione building-block supplement L-glutamine.

Glutathione which is stored in the liver can be made by the liver using the raw materials glycine, glutamine and cysteine. Because glutathione supplements are poorly absorbed, the present invention includes the raw materials L-glutamine and L-cysteine so the liver can manufacture it's own glutathione.

Vitamin C as calcium ascorbate, vitamin B-1 as thiamine and L-cysteine are also in the current formula as they are useful in combating the toxic effects of acetaldehyde. In a study, a group of rats were given a lethal dose, LD-90 of acetaldehyde large enough to kill 90% of them and the rats who took a cocktail of vitamin C, thiamine and cysteine survived while those without it died. These three supplements are scientifically proven to be effective in combating toxic levels of acetaldehyde and are as such included in this formula. Calcium ascorbate is used rather than ascorbic acid because it is less irritating to the stomach lining.

### REFERENCES

1. The Innoculated Mind, Edition: Oct. 28, 2003 http://www.ktkgalaxy.net/forb/mind/topic/topic2003 10 01.html
2. Michael W. King, Ph.D./IU School of Medicine http://www.dentistry leeds.ac.uk/biochem/thcme/glycolysis.html#ethanol
3. Clin Invest 83,314-6 5. Harada S et al (1981) Aldehyde dehydrogenase deficiency as a cause of facial flushing reaction to alcohol in Japanese
4. Survival Guide to the Holiday Season Steven Wm. Fowkes, http://www.ceri.com/holiday.htm
5. Effect of the Antioxidant 2″-O-Glycosylisovitexin from young green barley leaves on acetaldehyde formation in beer stored at 50 degree C. for 90 days. Nakajima, S., Hagiwara, Y., Hagiwara H., and Shibamoto, T. 1998, Journal of Agricultural and Food Chemistry, Vol. 46 (4): 1529-1531.
6. Effects of amino acids on acute alcohol intoxication in mice-concentrations of ethanol, acetaldehyde, acetate and acetone in blood and tissues. Arukoru Kenkyuto Yakubutsu Ison (JAPAN) October 1990, 25 (5) p429-40
7. Clinical Science (1999) 96, (549-555), Effects of adrenaline on glucose and glutamine metabolism and superoxide production by rat neutrophils http://cs.portlandpress.co.uk/cs/096/0549/cs0960549.htm
8. Res Commun Chem Pathol Pharmacol. January 1976;13(1):125-8 Protection against acute toxicity of acetaldehyde in mice. O'Neill P J, Rahwan R G.

1. A composition useful for preventing and treating the symptoms of alcohol hangover consisting of a biologically active supplement for oral administration comprising a therapeutically effective amount of a composition comprising:
(a) a succinic acid component;
(b) at least one component based on fumaric acid;
(c) at least one sub-component of the tripeptide glutathione;
(d) at least one component based on L-cysteine;
(e) at least one component based on young barley grass juice powder;
(f) a folic acid component;
(g) a vitamin B-12 component;
(h) a vitamin B-1 component; and
(I) at least one energy source
whereby said components are combined to generate a powder form or a liquid form compound, taken prior to, during, or after consumption of alcoholic beverages.

2. The composition useful for preventing and treating the symptoms of alcohol hangover according to claim 1, wherein said tripeptide glutathione sub-component is selected from the group of L-glutamic acid or L-glutamine.

3. The composition useful for preventing and treating the symptoms of alcohol hangover according to claim 1, wherein said component based on L-cysteine is selected from the group of L-cysteine, N-acetyl cysteine, or L-cysteine HCl.

4. The composition useful for preventing and treating the symptoms of alcohol hangover according to claim 1 wherein said succinic acid component includes succinic acid.

5. The composition useful for preventing and treating the symptoms of alcohol hangover according to claim 1, wherein said component based on fumaric acid is selected from the group of fumaric acid, ammonium monofumarate, ammonium difumarate, potassium difumarate, potassium monofumarate, sodium difumarate or sodium monofumarate.

6. The composition useful for preventing and treating the symptoms of alcohol hangover according to claim 1, wherein said component based on young barley grass juice powder is selected from the group of young barley grass juice powder, barley grass juice powder or barley grass powder.

7. The composition useful for preventing and treating the symptoms of alcohol hangover according to claim 1, wherein said vitamin B-12 is selected from the group of cyanocobalamin and cobalamin.

8. The composition useful for preventing and treating the symptoms of alcohol hangover according to claim 1, wherein said vitamin B-1 is selected from the group of thiamine hydrochloride and thiamine mononitrate.

9. The composition useful for preventing and treating the symptoms of alcohol hangover according to claim 1, wherein said energy source is selected from the group of dextrose, fructose, glucose or crystal glucose.

10. The composition useful for preventing and treating the symptoms of alcohol hangover according to claim 1, wherein said therapeutic composition is in the form of a liquid for oral administration to human individuals desiring relief from hangover symptoms.

11. The composition useful for preventing and treating the symptoms of alcohol hangover according to claim 1, wherein said therapeutic composition is in the form of a powder for oral administration to human individuals desiring relief from hangover symptoms.

12. The composition useful for preventing and treating the symptoms of alcohol hangover according to claim 1 1, wherein said therapeutic composition is in the form of a powder, and further in the form of a compressed tablet, for oral administration to human individuals desiring relief from hangover symptoms.

13. The composition useful for preventing and treating the symptoms of alcohol hangover according to claim 11, wherein said therapeutic composition is in the form of a powder, and further in the form of a powder filled capsule, for oral administration to human individuals desiring relief from hangover symptoms.

14. The composition useful for preventing and treating the symptoms of alcohol hangover according to claim 1, wherein said composition is comprised by weight of:

| | |
|---|---|
| a) L-glutamine | 10 to 400 mg.; |
| b) L-cysteine | 10 to 450 mg.; |
| c) fumaric acid | 10 to 400 mg.; |
| d) succinic acid | 10 to 400 mg.; |
| e) young barley grass juice powder | 10 to 400 mg.; |
| f) folic acid | 2 to 400 mcg.; |
| g) vitamin B-12 | 2 to 1200 mcg.; |
| h) vitamin B-1 | 2 to 50 mg.; |
| I) calcium ascorbate | 2 to 400 mg.; and |
| j) an energy source | 10 to 400 mg. |

15. The composition useful for preventing and treating the symptoms of alcohol hangover, wherein said composition is comprised by weight of:

| | |
|---|---|
| a) fumaric acid | 10 to 500 mg.; |
| b) succinic acid | 10 to 400 mg.; |
| c) L-glutamine | 10 to 500 mg.; |
| d) dextrose | 10 to 200 mg.; |
| e) sodium ascorbate | 10 to 200 mg.; |
| d) sodium benzoate | 10 to 100 mg.; |
| e) disodium phosphate | 10 to 100 mg.; |
| f) sodium citrate | 10 to 100 mg.; |
| g) sodium chloride | 10 to 100 mg. |
| h) potassium phosphate | 10 to 100 mg.; |
| I) monopotassium phosphate | 10 to 100 mg.; |
| j) sucrose acetate isobutyrate | 10 to 100 mg.; |
| k) niacinamide | 10 to 200 mg.; |
| l) pyridoxine hydrochloride | 10 to 200 mg.; and |
| m) purified water | 5 to 100 g. |

16. The composition useful for preventing and treating the symptoms of alcohol hangover according to claim 15, wherein said therapeutic composition is in the form of a liquid for oral administration to human individuals desiring relief from hangover symptoms.

17. The composition useful for preventing and treating the symptoms of alcohol hangover according to claim 15, wherein said therapeutic composition is in the form of a liquid for oral administration to human individuals desiring relief from hangover symptoms, and further administered orally in the dosage of one to two 1 oz. servings per two alcoholic beverages consumed.

18. The composition for treatment of an alcohol related hangover and method for using same according to claim 1, wherein the composition is administered orally to a person before consumption of alcohol.

19. The composition for treatment of an alcohol related hangover and method for using same according to claim 1, wherein the composition is administered orally to a person before and during consumption of alcohol.

20. The composition for treatment of an alcohol related hangover and method for using same according to claim 1, wherein the composition is administered to a person after consumption of alcohol.
